(19) United States
(12) Patent Application Publication
(10) Pub. No.: US 2008/0176937 A1
(43) Pub. Date: Jul. 24, 2008

(54) Novel crystalline mycophenolate sodium polymorph and processes to manufacture same

(75) Inventors: Chad Glass, Winnipeg (CA); Rudolf Kubela, Oakville (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, omitted

(73) Assignee: Apotex Fermentation Inc.

(21) Appl. No.: 11/790,100

(22) Filed: Apr. 24, 2007

Related U.S. Application Data
(63) Continuation-in-part of application No. 11/657,465, filed on Jan. 25, 2007.

(30) Foreign Application Priority Data
Jan. 23, 2007 (CA) 2,574,940
Apr. 20, 2007 (CA) 2,585,601

(51) Int. Cl. A61K 31/335

(52) U.S. Cl. 514/469; 549/469

(57) ABSTRACT

The present invention includes the discovery of a novel polymorphic form of sodium mycophenolate (MPS) and processes for its formation. Furthermore, the present invention also includes a process to efficiently and cleanly convert the novel polymorphic form to known Form M2.

This is as a Continuation-In-Part of U.S. patent application Ser. No. 11/657,465 filed Jan. 25, 2007.

### TITLE OF INVENTION

A novel crystalline mycophenolate sodium polymorph and processes to manufacture same.

### FIELD OF INVENTION

The present invention relates to a new polymorphic form of mycophenolate sodium.

### BACKGROUND OF THE INVENTION

This invention relates to a new polymorphic form of mycophenolate sodium salt, preparations and uses thereof.

Mycophenolic acid was first isolated in 1896 and has been extensively investigated as a pharmaceutical drug of commercial interest. It is known to have anti-tumor, anti-viral, immunosuppressive, anti-psoriatic, and anti-inflammatory activity [see e.g. W. A. Lee et al, Pharmaceutical Research (1990), 7, p. 161-166 and references cited therein].

The sodium salt of mycophenolic acid (depicted below) has been employed as inhibitors of malignant tumor growth in mammals for a half century. Previously, Eli Lilly revealed the inhibiting effect of mycophenolate sodium salt (MPS) on the growth of tumors in mammals [see M. J. Sweeney et al., Cancer Research (1972), 32, p. 1795-1802]. Currently, Novartis markets an enteric-coated formulation of MPS [see WO 97/38689] under the name Myfortic® as an immunosuppressant medicine for organ transplant recipients.

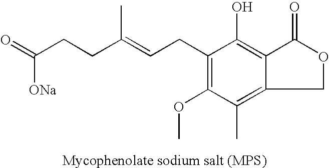

South African patent No. 6804959 describes the formation of MPS by dissolving the corresponding mycophenolic acid in chloroform, followed by addition of an anhydrous methanol solution of sodium methoxide and later recrystallization from n-pentane. When this method was repeated by Molnar et al (US2006/0069152 A1) the polymorphic form produced was identified as Form M2.

J. Med. Chem. (1996), 39, 1236-1242 describes treating a solution of mycophenolic acid in ethanol with equimolar sodium ethoxide at room temperature. The desired salt was recovered after solvent removal under vacuum. When this method was repeated by Molnar et al (US2006/0069152 A1) the polymorphic form produced was identified as Form M2.

PCT 97/38689 discloses a synthetic route to MPS that is identical to that of ZA No. 6804959. It further describes a process for recrystallizing the sodium salt from an acetone/ethanol mixture or, if necessary, water. The melting point of the obtained salt product is 189-191° C.

Acta Crystallographica Sect. C, (2000), C56, p. 432-433 describes a process for producing MPS. A methanolic solution of mycophenolic acid was treated with 1 equivalent of sodium methoxide. After 1 hour of stirring, the solvent was removed by evaporation to give a crystalline solid (mp 190° C.). Single crystal samples were grown by dissolving MPS in water/ethyl acetate mixture at 50° C. and then cooling the resulting solution to room temperature. A single crystal X-ray structure of the produced polymorph is also disclosed. When this method was repeated by Molnar et al (US2006/0069152 A1) the polymorphic form produced was identified as a mixture of Forms M2 and M3.

PCT 2006/012379 describes multiple processes for the formation of the M2 polymorphic form of MPS. In certain examples, mycophenolic acid is dissolved in varying solvents before treatment with the sodium bases. After stirring the precipitated product is filtered off and washed with cooled solvent. Also, this application discloses the dissolution of MPS in various organic solvents heated to elevated temperatures before cooling and recovery of crystalline MPS Form M2.

PCT 2004/020426 describes processes for producing mycophenolate sodium. In this work, mycophenolic acid or its ammonium salt is dissolved in ethyl acetate solution before addition of a sodium salt of an alkyl carboxylic acid. The desired MPS is recovered in crystalline form upon chilling of the solution.

US Patent Application No. 2006/0069152 describes the processes for the formation of a number of polymorphic forms of MPS, specifically M1-12, M15-22, and M26-28. X-ray diffraction (XRD) and differential scanning calorimetry (DSC) data are provided for the novel polymorph. Data for a small number of polymorphic disodium salt forms are also provided.

The discovery of new polymorphic forms of a pharmaceutically useful compound and/or new processes for their preparation provides a new opportunity to improve the performance profile of a pharmaceutical product. It widens the scope of materials that a drug formulator has available for designing, for example, a pharmaceutical dosage form of a drug with a specific bioavailability profile or other desired characteristics.

There are ever increasing demands on the efficiency of reactions used to form pharmaceutically active products. Higher yielding reactions are clearly more economical from a financial point of view. Furthermore, reactions that generate products of higher purity are highly desirable because they minimize formation of unwanted and potentially harmful impurities.

### SUMMARY OF THE INVENTION

The present invention encompasses a novel polymorphic form of monosodium mycophenolate, denominated CG1, the manufacture of CG1 and uses thereof.

In one embodiment, the present invention encompasses a polymorphic form of crystalline MPS denominated Form CG1. Form CG1 is an anhydrous form of the mono-sodium salt. Form CG1 is characterized by a powder x-ray diffraction (XRD) pattern with peaks at 4.6, 5.2, 6.1, 7.2, 10.5, 12.4, 14.4, 17.1, 22.9, 24.4, 25.2, 26.6, 26.9±0.2 degrees 2 theta (FIG. 1) and/or Fourier Transform-Infrared (FT-IR) spectrum with peaks at 2924, 2854, 1719, 1563, 1461, 1377, 1266, 1135, 1078, 1034 wavenumbers (FIG. 2). Form CG1 may be further characterized by a Differential Scanning Calorimetry (DSC) curve (FIG. 3).

One process for preparing crystalline MPS Form CG1 comprises preparing a suspension of mycophenolic acid in water; combining an aqueous sodium inorganic base solution, preferably selected from the group consisting of aqueous NaOH, aqueous NaHCO₃, aqueous Na₂CO₃, and aqueous NaOAc, to obtain an aqueous MPS solution; adding an organic solvent, preferably toluene, that forms an azeotrope with water; azeotropically removing the water by heating to reflux, preferably with a Dean-Stark apparatus; isolating the crystalline form from the remaining organic media at high temperature, preferably via crystallization or precipitation; and recovering the crystalline form.

In another embodiment, the present invention encompasses converting the novel polymorphic form of crystalline MPS (CG1), into known polymorphic Form M2 by heating a suspension of CG1 in toluene to reflux, for a predetermined time in order to convert CG1 to M2, and recovering the crystalline form. The resulting Form M2 material is recovered in quantitative yield and with no detectable impurities.

In one embodiment there is provided a crystalline mycophenolate sodium salt (Form CG1) characterized by a powder XRD pattern with peaks at 4.6, 5.2, 6.1, 7.2, 10.5, 12.4, 14.4, 17.1, 22.9, 24.4, 25.2, 26.6, 26.9±0.2 degrees 2 theta.

In another embodiment there is provided a crystalline mycophenolate sodium salt (Form CG1) characterized by a FT-IR pattern with peaks at 2924, 2854, 1719, 1563, 1461, 1377, 1266, 1135, 1078, 1034 wavenumbers.

In yet another embodiment there is provided a crystalline mycophenolate sodium salt (Form CG1) characterized by a DSC as shown in FIG. 3.

There is further provided a process for preparing crystalline mycophenolate sodium salt (Form CG1) comprising the steps of:
(a) preparing a suspension of mycophenolic acid in water;
(b) addition of an aqueous solution of a sodium inorganic base, preferably selected from the group preferably selected from the group consisting of aqueous NaOH, aqueous NaHCO₃, aqueous Na₂CO₃, and aqueous NaOAc to obtain an aqueous mycophenolate sodium salt solution;
(c) addition of an organic solvent, preferably toluene, that forms an azeotrope with water to the aqueous mycophenolate sodium salt solution;
(d) azeotropic removal of water by heating to reflux resulting in the crystalline Form CG1; and
(e) recovering the crystalline form wherein Form CG1 is characterized by at least one of the following:
   i) a powder XRD pattern with peaks at 4.6, 5.2, 6.1, 7.2, 10.5, 12.4, 14.4, 17.1, 22.9, 24.4, 25.2, 26.6, 26.9±0.2 degrees 2 theta;
   ii) a FT-IR pattern with peaks at 2924, 2854, 1719, 1563, 1461, 1377, 1266, 1135, 1078, 1034 wavenumbers; or
   iii) a DSC as shown in FIG. 3.

In yet another embodiment there is provided a process for preparing crystalline mycophenolate sodium salt (Form M2) comprising the steps of:
(a) preparing a suspension of crystalline mycophenolate sodium salt (Form CG1) in toluene;
(b) heating said suspension at reflux;
(c) crystallizing the crystalline form; and
(d) recovering the crystalline Form M2.

Preferably Form CG1 is characterized by a powder XRD pattern with peaks at 4.6, 5.2, 6.1, 7.2, 10.5, 12.4, 14.4, 17.1, 22.9, 24.4, 25.2, 26.6, 26.9±0.2 degrees 2 theta.

Preferably Form CG1 is also characterized by a FT-IR pattern with peaks at 2924, 2854, 1719, 1563, 1461, 1377, 1266, 1135, 1078, 1034 wavenumbers.

Preferably Form CG1 is also characterized by a DSC as shown in FIG. 3.

### BRIEF DESCRIPTION OF THE DRAWINGS

The following figures illustrate preferred and alternative embodiments of the invention, wherein:

FIG. 1 is a characteristic x-ray powder diffraction pattern for monosodium mycophenolate Form CG1.

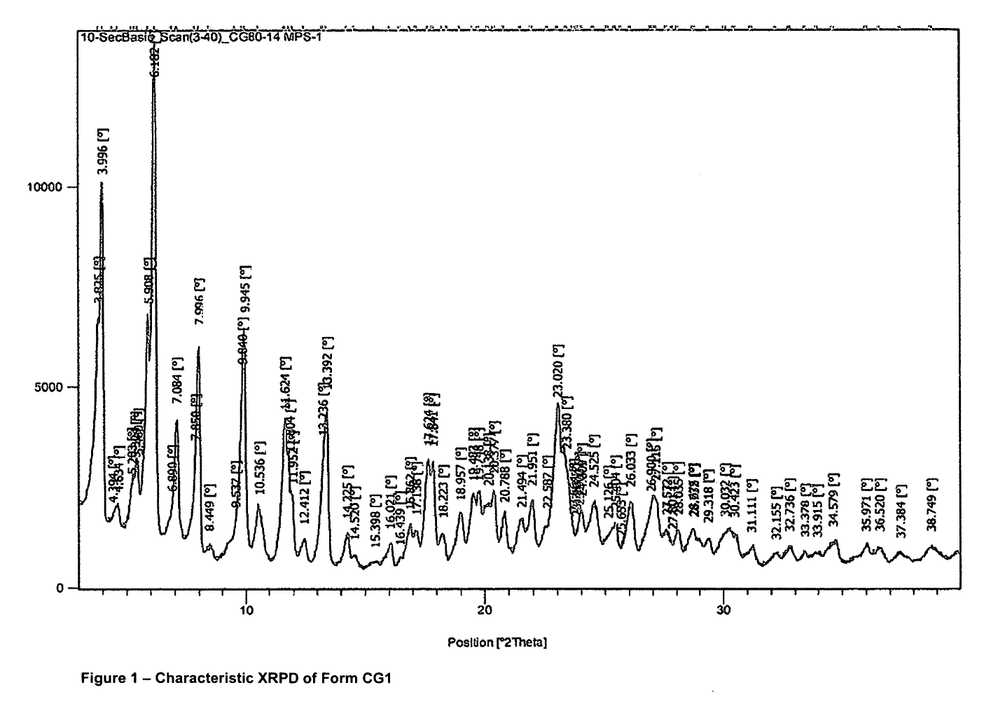

FIG. 2 is a characteristic FT-IR spectrum of monosodium mycophenolate Form CG1.

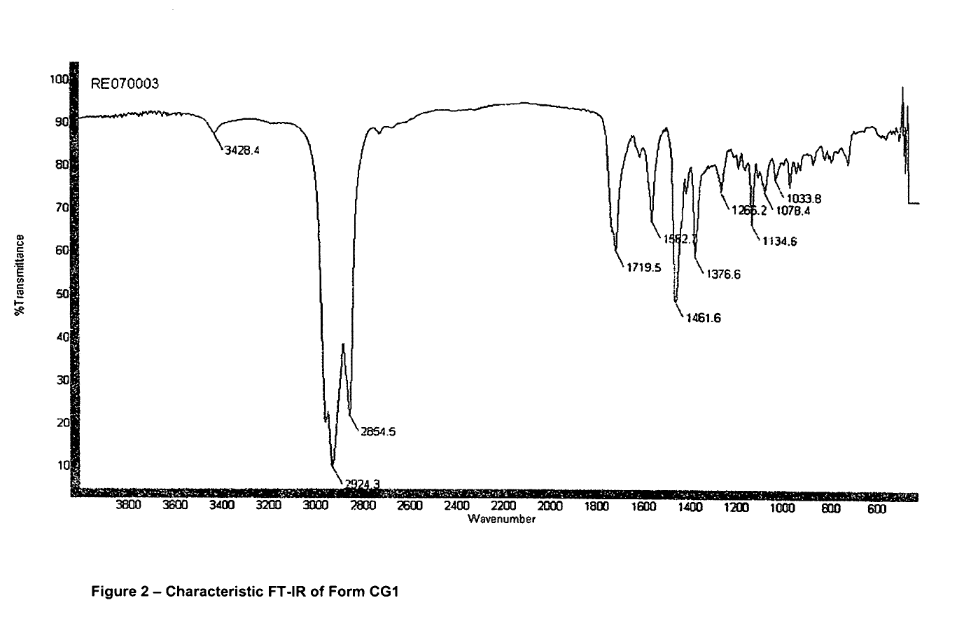

FIG. 3 is a characteristic DSC curve for monosodium mycophenolate Form CG1.

### DETAILED DESCRIPTION OF THE INVENTION

The following examples are for the preparation of MPS polymorphic Form CG1.

### EXAMPLE 1

To a stirred suspension of mycophenolic acid (20 g) in water (50 mL), one molar equivalent of 50% aqueous NaOH (3.2 mL) was added. When all of the acid had been utilized and dissolved, toluene (360 mL) was added and the reaction mixture was heated to reflux and the water was azeotropically removed with the help of a Dean-Stark apparatus until the reaction mixture reached 110-111° C. The precipitation of the MPS started at 105° C. The mixture was cooled to room temperature and the crystalline material was recovered by filtration. The solid MPS Form CG1 was dried at 50±5° C. in a vacuum oven.

### EXAMPLE 2

MPS (2 g) was dissolved at room temperature in water (10 mL) and toluene (36 mL) was added to the solution. The solution was heated to reflux and the water was azeotropically removed using a Dean-Stark apparatus until the mixture reached 110-111° C. The precipitation of the MPS began at 105° C. The mixture was cooled to room temperature and the crystalline material was recovered by filtration. The solid MPS Form CG1 was dried at 50±5° C. in a vacuum oven.

The following example is for the preparation of MPS polymorphic Form M2 from Form CG1.

### EXAMPLE 3

MPS (2 g) Form CG1 was added to toluene (36 mL). The resulting suspension was heated at reflux for preferably 24 hours. The resulting crystalline MPS material was recovered by filtration. The solid MPS was dried at 50±5° C. in a vacuum oven. The crystalline material formed, in quantitative yield and with no additional impurities, was determined to be polymorphic Form M2 (the most thermally stable form).

While the foregoing provides a detailed description of a preferred embodiment of the invention, it is to be understood that this description is illustrative only of the principles of the invention and not limitative. Furthermore, as many changes can be made to the invention without departing from the scope of the invention, it is intended that all material contained herein be interpreted as illustrative of the invention and not in a limiting sense.

The embodiments of the invention in which an exclusive property or privilege is claimed are as follows:

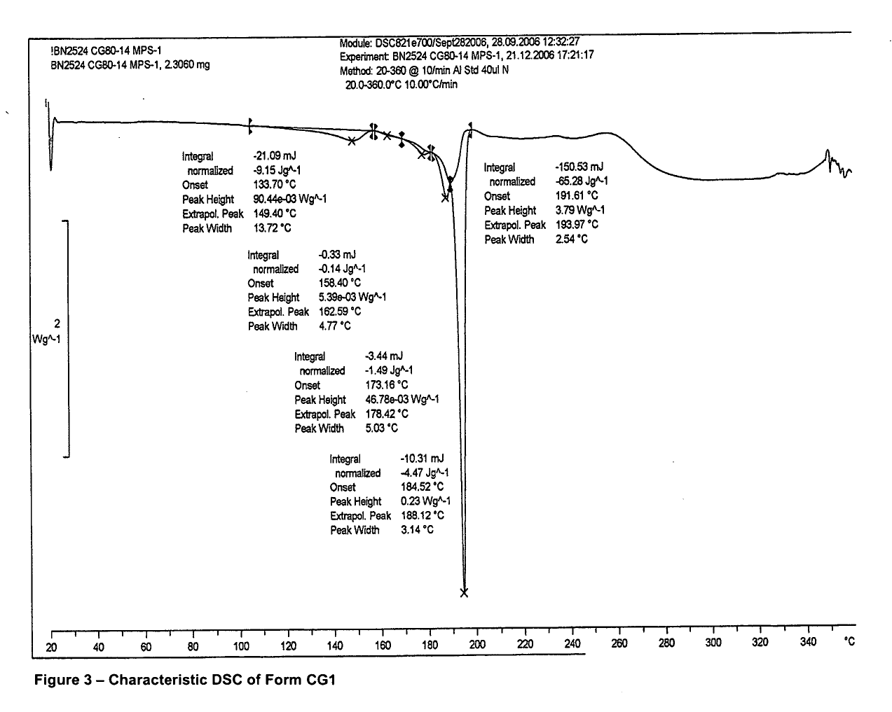

1. A crystalline mycophenolate sodium salt (Form CG1) characterized by at least one of the following:
i) a powder XRD pattern with peaks at 4.6, 5.2, 6.1, 7.2, 10.5, 12.4, 14.4, 17.1, 22.9, 24.4, 25.2, 26.6, 26.9±0.2 degrees 2 theta;
ii) a FT-IR pattern with peaks at 2924, 2854, 1719, 1563, 1461, 1377, 1266, 1135, 1078, 1034 wavenumbers;
iii) a DSC as shown in FIG. 3.

2. A process for preparing crystalline mycophenolate sodium salt (Form CG1) comprising the steps of:
(a) preparing a suspension of mycophenolic acid in water;
(b) addition of an aqueous solution of a sodium inorganic base to obtain an aqueous mycophenolate sodium salt solution;
(c) addition of an organic solvent that forms an azeotrope with water to the aqueous mycophenolate sodium salt solution;
(d) azeotropic removal of water by heating to reflux resulting in the crystalline Form CG1; and
(e) recovering the crystalline form wherein Form CG1 is characterized by at least one of the following:
i) a powder XRD pattern with peaks at 4.6, 5.2, 6.1, 7.2, 10.5, 12.4, 14.4, 17.1, 22.9, 24.4, 25.2, 26.6, 26.9±0.2 degrees 2 theta;
ii) a FT-IR pattern with peaks at 2924, 2854, 1719, 1563, 1461, 1377, 1266, 1135, 1078, 1034 wavenumbers; or
iii) a DSC as shown in FIG. 3.

3. The process of claims 1 or 2, wherein the organic solvent is toluene.

4. A process for preparing crystalline mycophenolate sodium salt (Form M2) comprising the steps of:
(a) preparing a suspension of crystalline mycophenolate sodium salt (Form CG1) in toluene;
(b) heating said suspension at reflux;
(c) crystallizing the crystalline form; and
(d) recovering the crystalline Form M2.

5. The process of claim 4 wherein the Form CG1 is characterized by at least one of the following:
i) a powder XRD pattern with peaks at 4.6, 5.2, 6.1, 7.2, 10.5, 12.4, 14.4, 17.1, 22.9, 24.4, 25.2, 26.6, 26.9±0.2 degrees 2 theta;
ii) a FT-IR pattern with peaks at 2924, 2854, 1719, 1563, 1461, 1377, 1266, 1135, 1078, 1034 wavenumbers; or
iii) a DSC as shown in FIG. 3.

6. The crystalline mycophenolate sodium salt (Form CG1) of claim 1 wherein the salt is mono sodium.

7. The crystalline mycophenolate sodium salt (Form CG1) of claim 1 or 6 wherein the salt is anhydrous.

8. The process of claims 2, 4 or 5 wherein Form CG1 is anhydrous.

9. The process of claims 2, 4, or 5 wherein Form CG1 is a monosodium salt.
